(19) United States
(12) Patent Application Publication
(10) Pub. No.: US 2009/0096260 A1
(43) Pub. Date: Apr. 16, 2009

(54) Kozy baby

(76) Inventors: Cheryl Ann Long, Aurora, CO (US); Fermie Boatner, Denver, CO (US); Robbin Traore, Centennial, CO (US); Keshawn Houston, Longmont, CO (US); Javetta Giles, Aurora, CO (US)

Correspondence Address: Cheryl Ann Long, P.O. Box 110494, Aurora, CO, 80042, US

(21) Appl. No.: 11/900,011

(22) Filed: Oct. 10, 2007

(51) Int. Cl. A47C 31/00; A61F 7/00

(52) U.S. Cl. 297/219.12; 607/114

(57) ABSTRACT

An efficient way to cool or warm the body of children while using a car seat, stroller, or baby swing when the surrounding temperature is not comfortable. It is body pillow that is shaped to fit into the car seat, stroller, or baby swing that has three rectangular pockets located on the back of it. These pockets are where cooling/warming material, also known as the non-toxic gel pack, is inserted. The body of the child will lye against this pillow with the cooling/warming material, also known as the non-toxic gel pack, inserted into the pockets, while properly seated in the car seat, stroller or baby swing. With the use of this invention, the child's body will be quickly cooled or warmed. Neck support is also provided with the use of an additional pillow if needed for smaller children.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not applicable.

### BACKGROUND OF THE INVENTION

1. Technical Field

This invention is a cooling or warming method for children to use while they are properly seated in a car seat, stroller, or baby swing etc. This invention is a removable body pillow that the body of the child will lye against while they are properly in the car seat, stroller etc. The purpose of this invention is to warm or cool the body of the child when surrounding temperatures are not favorable. Children can easily overheat when the surrounding temperature is very hot, or become very cold when the surrounding temperatures are quite cold. This invention will cool down the body of the child, or warm the body of the child in those situations without the use of an air conditioner or heater. If this invention were to be sold in a store, it would be in the infant and toddler aisle.

2. Background Art

Car seats, strollers, and baby swings are all products that are used on a daily basis by many people. This invention was created to assist in the regulation of the child's body temperature when products such as those listed above are used and surrounding temperatures are not favorable. There are many inventions that were previously made to cool or warm up a car seat, but the product was removed before the child was actually seated. Products such as those will work for a short time, but if the child is in the seat for a long time, the warming or cooling may wear off and the child will once again, become uncomfortable. These products were intended to cool or warm the seat initially when the child is put into the car seat when, for example, the car has been sitting outside in very hot temperatures, and the car seat and or buckle is very hot to touch. Kozy Baby was invented to constantly regulate the temperature of the child, not the seat, so the child is comfortable the entire time while seated in the car seat, stroller, or baby swing. This invention stays in the seat with the child the entire time the child is seated. This invention is easily removable though, so it is quickly interchangeable between the car seat, stroller, or baby swing. The temperature comfort “goes” with the child.

### BRIEF SUMMARY OF THE INVENTION

This invention is a body pillow that is shaped to fit into a car seat, stroller, or baby swing. This invention is intended for children to use while they are seated in the car seat, stroller, or baby swing. This pillow has three rectangular-shaped pockets on the back of it where cooling/warming material, also known as non-toxic gel packs, are inserted. The body of the child will lye against the pillow while they are properly seated in the car seat, stroller, or baby swing. This invention also includes a neck support pillow that can be easily attached or removed if needed for smaller children. This invention is easily inserted into the car seat, stroller, or baby swing and is just as easily removed so it can be used in many different seats. The purpose of this invention is to cool or warm the body of the child when surrounding temperatures are not favorable, such as being in really hot or cold weather.

There are many problems that this invention would solve. They are as follows:
It is too hot in the vehicle because there is not an air conditioner or the air conditioner is not functional.
Even with the use of an air conditioner, the cool air may not reach to the back of the vehicle quickly where the child is seated, or not at all.
Children can overheat very quickly in a hot vehicle, can get a heat rash, or even die in extreme cases.
It can become very expensive to use the air conditioner.
When out on a walk outside and the temperature is very hot, the child can overheat and be very uncomfortable.
There is no time to “warm” up the vehicle when it is cold outside and one needs to take their child with them.
It is too cold in the vehicle because the heater is not functional.
Even with the use of a heater, the warm air may not reach the back of the vehicle quickly where the child is seated, or not at all.
When out on a walk outside, it may be very cold outside, and one needs to warm their child to keep them comfortable.
Sometimes to keep the child warm, the child needs to be bundled in a lot of clothing and they will not comfortable fit in the car seat or stroller.

With the use of this invention, the problems listed above can be solved without the use of an air conditioner or a heater, which could be very expensive.

### BRIEF DESCRIPTION OF THE SEVERAL VIEWS OF THE DRAWING(S)

FIG. 1 shows a frontal view of Kozy Baby. FIGS. 1A through 1J show all the different frontal pieces of Kozy Baby that when put together create the full frontal view. FIGS. 1K and 1L represent pieces that are needed when using Kozy Baby, but cannot be seen from the frontal view.

FIG. 1: This is the full frontal view of Kozy Baby.

FIG. 1A: This is the frontal view of the top part of Kozy Baby. The cuts on the sides represent the area where ‘A’ lines will be put into place. The ‘A’ lines will create the area where the seat belts of the car seat thread through.

FIG. 1B: This represents a piece of material that is connected to the top part of FIG. 1A. There is also a piece of fastening tape, also known as Velcro, that is located on the top, middle part of this figure.

FIG. 1C: This is the frontal view of the middle part of Kozy Baby.

FIG. 1D: This is the frontal view of the bottom part of Kozy Baby.

FIG. 1E: This is the neck support pillow that attaches to the top part of FIG. 1A, underneath FIG. 1B though.

FIG. 1F: This represents the frontal view of the side trimming that goes on the left side of Kozy Baby.

FIG. 1G: This represents the frontal view of the side trimming that goes on the right side of Kozy Baby.

FIG. 1H: This represents the two frontal views of the top trimming that goes around the left and right top parts of FIG. 1A.

FIG. 1I: This figure represents the frontal view of the two pieces that go on the two cut sides of FIG. 1A. They are ‘A’ lines that are slots where the belt of a car seat thread through.

FIG. 1J: This represents the pieces of fastening tape that go on the ‘A’ lines of FIG. 1I. The fastening tape closes the openings to secure the seat belts in place.

FIG. 1K: This represents the piece of fastening tape that is put onto the car seat or stroller. When Kozy Baby is in use, this figure is connected to the fastening tape described in FIG. 1B.

FIG. 1L: This represents a piece of fastening tape that is used to put between FIGS. 1B and 1K. The purpose of this piece of fastening tape is to make Kozy Baby adjust to the different sizes of car seats and strollers.

FIG. 1M: This represents a slip cover that is put over the cooling/warming material, also known as the non-toxic gel pack.

FIG. 2 represents the full back view of Kozy Baby. When FIGS. 2A though 2I are put together, they create the full back view.

FIG. 2A: This represents the top part of the back of Kozy Baby.

FIG. 2B: This represents the middle part of the back of Kozy Baby.

FIG. 2C: This represents the bottom of the back part of Kozy Baby. The square in the middle represents where the seat buckle of a car seat or stroller pulls through.

FIG. 2D: This represents the 3 ‘A’ lines that are on the top (FIG. 2A), the middle (FIG. 2B), and the bottom (FIG. 2C) back parts of Kozy Baby. These ‘A’ lines create the pockets where the cooling/warming material, also known as the non-toxic gel packs, go into.

FIG. 2E: This represents trimming that goes under the first ‘A’ line that is on the top part of the back of Kozy Baby.

FIG. 2F: This represents trimming that goes under the second ‘A’ line that is on the middle part of the back of Kozy Baby.

FIG. 2G: This represents the back view of the two pieces of the top trimming that goes around the left and right top parts of FIG. 2A.

FIG. 2H: This represents the back view of the side trimming that goes around the left side of Kozy Baby.

FIG. 2I: This represents the back view of the side trimming that goes on the right side of Kozy Baby.

FIG. 2J: This represents the back view of the ‘A’ lines where the seat belts of a car seat thread through.

### DETAILED DESCRIPTION OF THE INVENTION

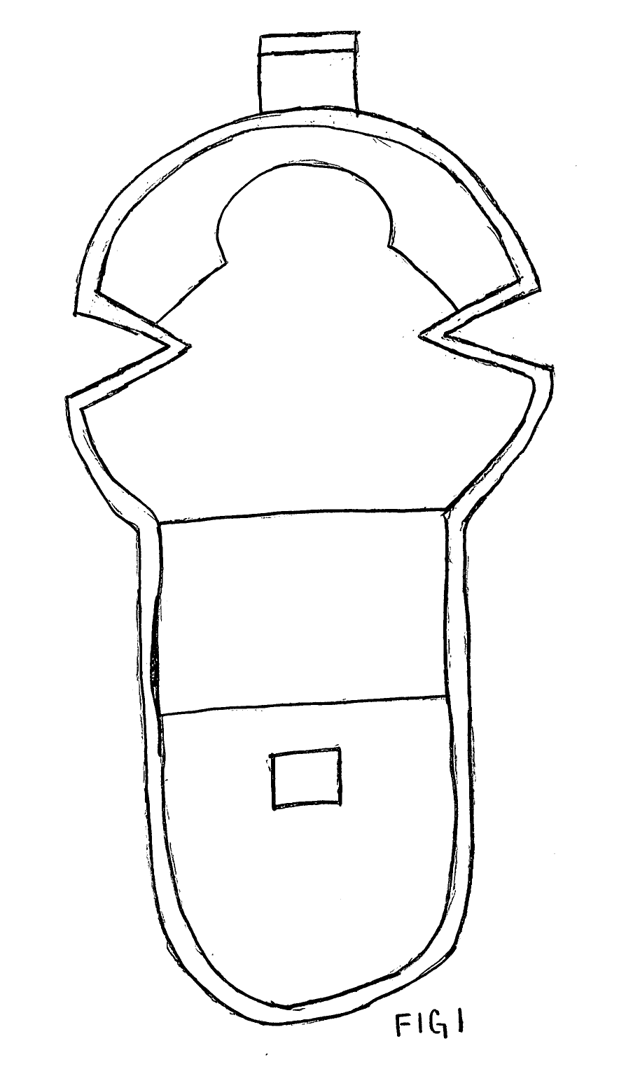

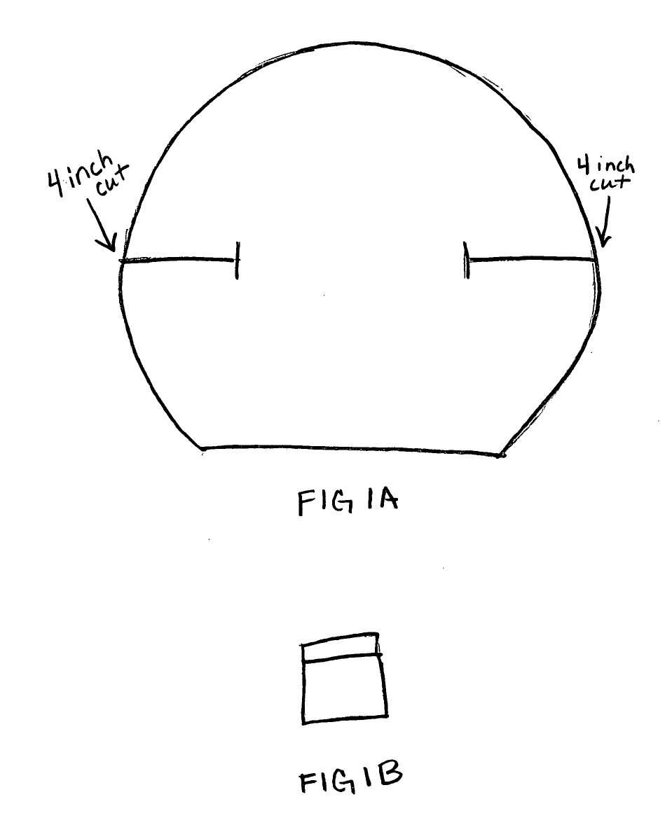

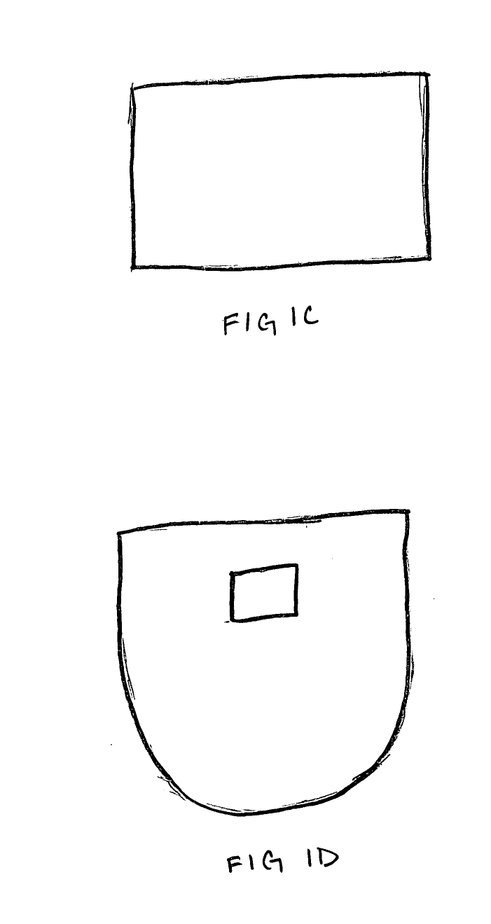

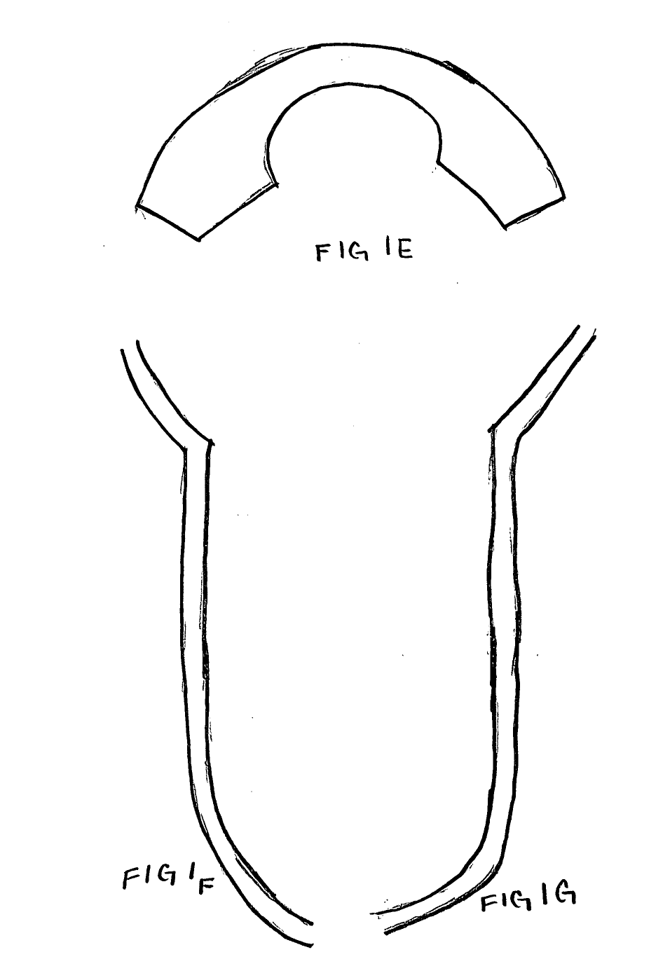

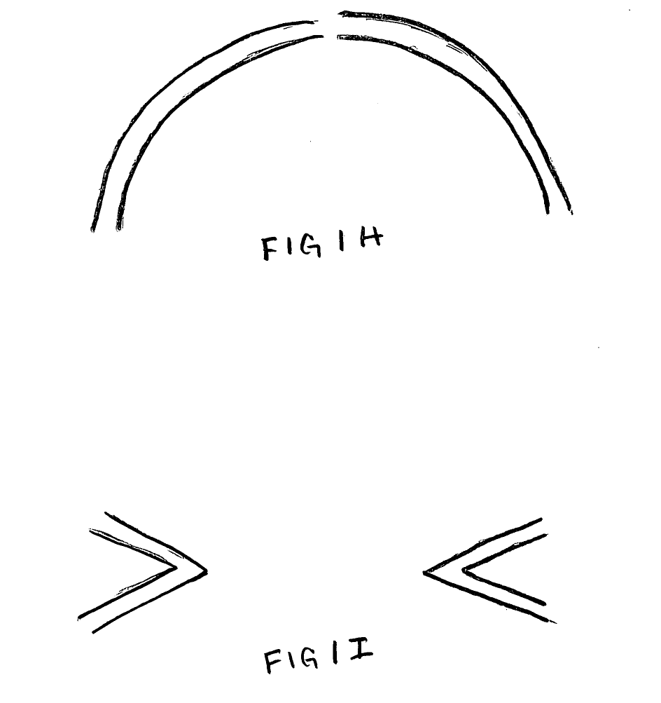

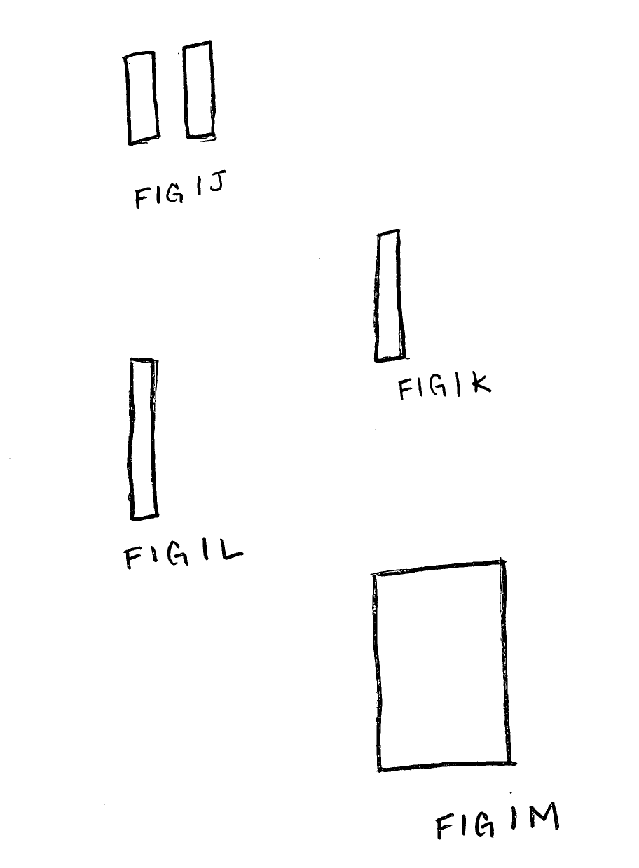

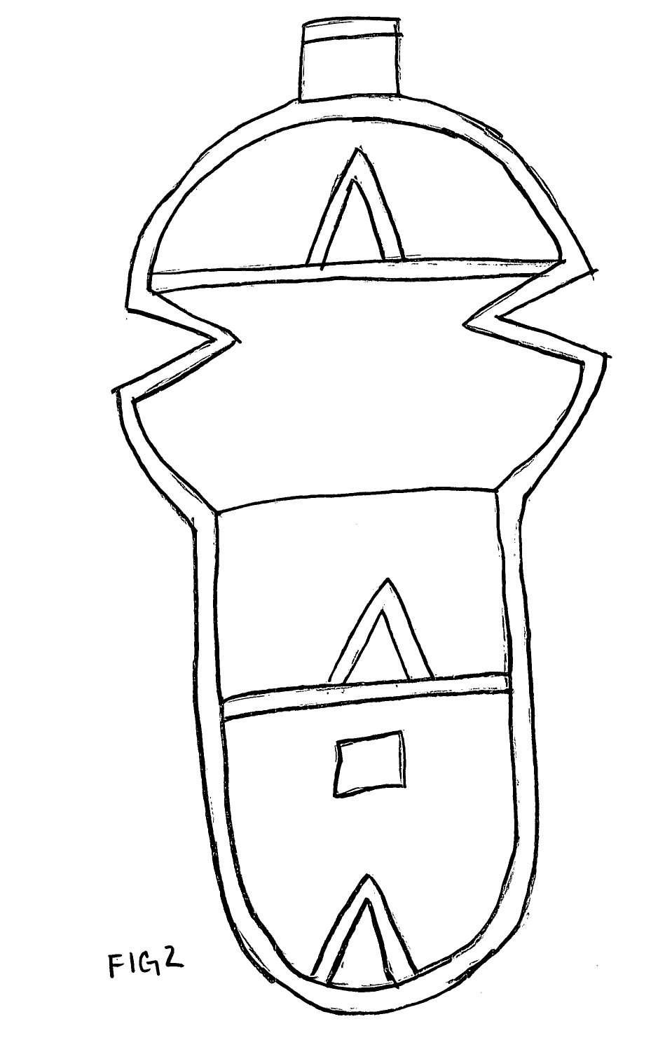

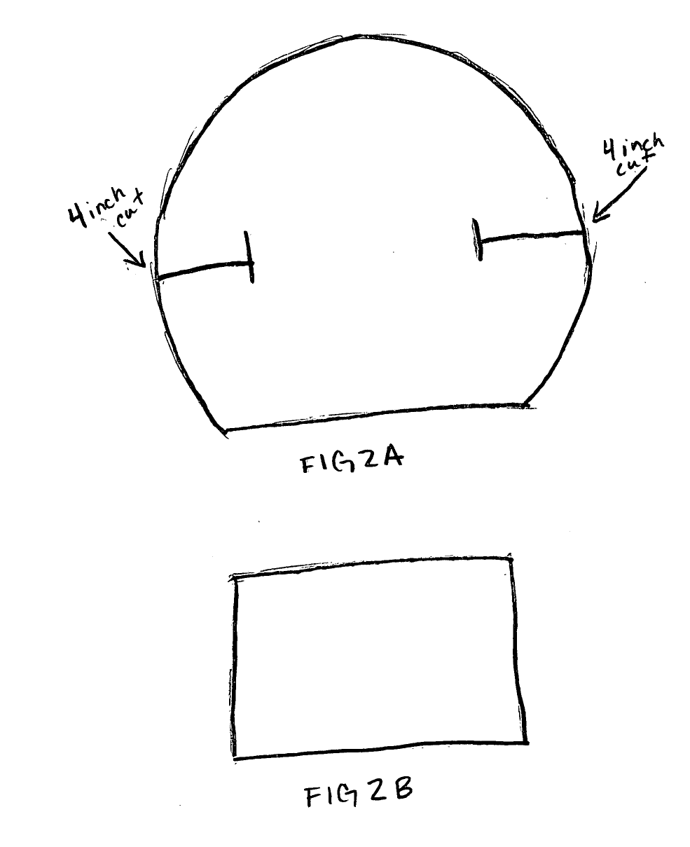

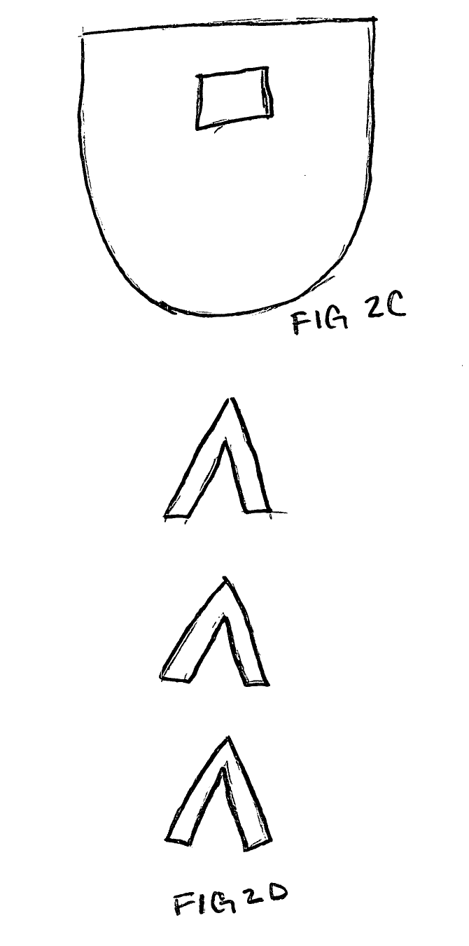

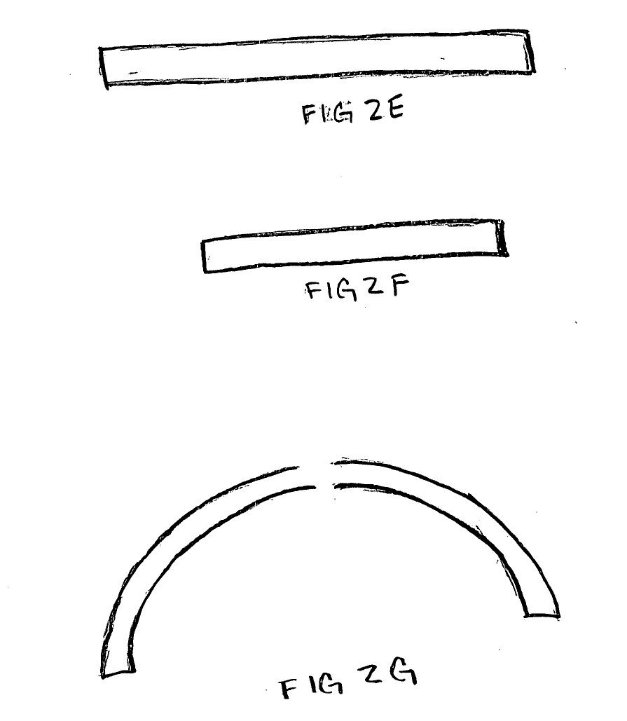

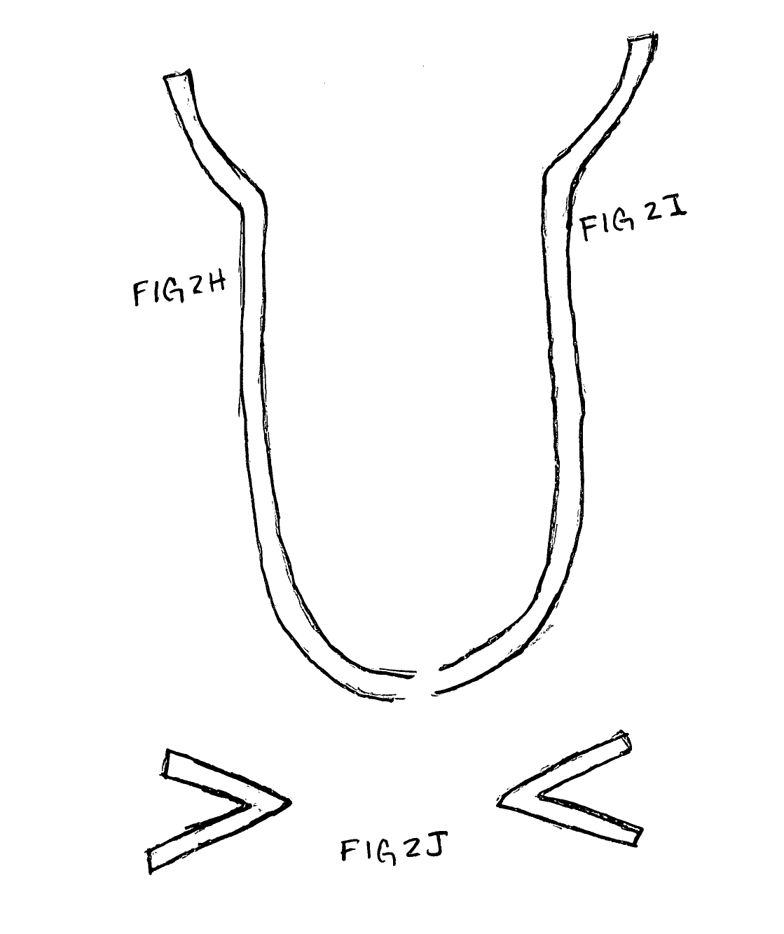

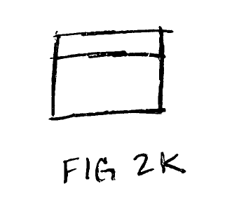

Kozy Baby is pillow that is put into a car seat, stroller, or baby swing that the child, while properly seated in those listed above, will lye against while the car seat, stroller, or baby swing is in use. The main purpose of this invention is to cool or warm the body of the child while they are seated in a car seat, stroller, or baby swing, and surrounding temperatures are not favorable. This invention also provides neck support when needed for smaller children. This invention is made out of fabric. Several different pieces, shown in the FIGS. 1A through 1M and FIGS. 2A through 2K, are sewn together to create the final product.

FIG. 1A represents where the child will rest their head against. It is the top frontal part of Kozy Baby. It is approximately 22 inches vertically and approximately 18 inches horizontally. The 4 inch cuts that are on the sides of this figure were created to have a place for the seat belts to thread through when Kozy Baby is used in a car seat.

FIG. 1B represents a piece of material that is sewn to the top of FIG. 1A. It is positioned in the middle. It is approximately 9 and a half inches vertically and approximately 4 and a half inches horizontally. There is also a piece of fastening tape, also known as Velcro, that is at the top of this figure. It is approximately 4 and a half inches horizontally and approximately 1 and a half inch vertically. The purpose of the fastening tape is so Kozy Baby will stay attached to and in place when in a car seat or stroller. The piece of fastening tape described above will connect to FIG. 1K.

FIG. 1C represents the frontal middle part of Kozy Baby. It is approximately 6 inches vertically and approximately 14 inches horizontally. The child's back will rest against this part when properly seated in the car seat, stroller, or baby swing. The top part of the figure is sewn to the bottom part of FIG. 1A. The bottom part is sewn to the top part of FIG. 1D.

FIG. 1D represents the frontal bottom part of Kozy Baby. Is it approximately 11 and a half inches vertically and approximately 14 inches horizontally. The cut in the middle that looks like a square is where the car seat or stroller buckle pulls through. This “square” is approximately 3 and a half inches by approximately 3 and a half inches. The frontal bottom part of Kozy Baby is where the child's feet and legs will lye against. The top part of this figure is sewn to the bottom part of FIG. 1C.

FIG. 1E represents a pillow that can be attached with fastening tape to FIG. 1A. This is a pillow that is used to support the neck of smaller children. It can be easily detached when an older child uses Kozy Baby. It is approximately 26 inches long and 8 inches wide. It is stuffed with polyester felt.

FIG. 1F represents side trimming that is sewn to the left sides of FIG. 1C and FIG. 1D. It is approximately 19 inches long and approximately 1 inch wide.

FIG. 1G represents side trimming that is sewn to the right sides of FIG. 1C and FIG. 1D. It is approximately 19 inches long and approximately 1 inch wide.

FIG. 1H represents the two pieces of top trimming that is sewn to the top left and top right parts of FIG. 1A. They are both approximately 19 inches long and approximately 1 inch wide.

FIG. 1I represents two ‘A’ lines that are sewn onto the 2 cut parts of FIG. 1A. The material used to create the ‘A’ line is approximately 10 inches long and approximately 1 inch wide, but once the ‘A’ line is put into place, the opening itself is approximately 4 inches. These ‘A’ lines create the area where the seat belts of a car seat will thread through.

FIG. 1J represents the pieces of fastening tape that are connected to the figures represented in FIG. 1I. The fastening tape is needed to secure the seat belts that are threaded through the ‘A’ lines shown on FIG. 1I to keep them into place. The pieces are approximately 3 and a half inches long and approximately 1 inch wide.

FIG. 1K represents a piece of fastening tape that is put onto the car seat or stroller. This piece of fastening tape is needed to keep Kozy Baby attached to the seat. It is approximately 4 and a half inches horizontally and approximately 1 and a half inch vertically.

FIG. 1L represents another piece of fastening tape that is used to put between FIGS. 1B and 1K to make Kozy Baby adjust to fit different styled and sized car seats and strollers. It is approximately 4 and a half inches horizontally and approximately 5 inches vertically.

FIG. 1M represents a slip cover, also known as batting, that can be put around the cooling/warming material, also known as the non-toxic gel pack.

FIG. 2 represents the full back view of Kozy Baby.

FIG. 2A represents the top of the back part of Kozy Baby. The two lines on both the left and right side represent two cuts that are approximately 4 inches in length.

FIG. 2B represents the middle of the back part of Kozy Baby. It is approximately 6 inches vertically and approximately 14 inches horizontally. The top of this figure is sewn to the bottom of FIG. 2A. The bottom of this figure is sewn to the top of FIG. 2C.

FIG. 2C represents the bottom part of the back of Kozy Baby. It is approximately 11 and a half inches vertically and approximately 14 inches horizontally. The square in the middle represents where the seat buckle of a car seat or stroller pulls through. The square is approximately 3 and a half inches by approximately 3 and a half inches. The top part of this figure is sewn to the bottom part of FIG. 2B.

FIG. 2D represents the 3 ‘A’ lines that are sewn on the top back, middle back, and bottom back parts of Kozy Baby. These ‘A’ lines create the opening to the pockets where the cooling/warming material, also known as non-toxic gel packs, are inserted. They are each made from material that is approximately 15 inches long, and approximately 1 inch wide, but when sewn in properly to create the ‘A’ line, the opening is approximately 7 inches from the top to the bottom.

FIG. 2E represents trimming that is sewn under the top ‘A’ line shown in FIG. 2D. It is approximately 18 inches long and approximately 1 inch wide.

FIG. 2F represents trimming that is sewn under the middle ‘A’ line that is shown in FIG. 2D. It is approximately 14 inches long and approximately 1 inch wide. The purpose of the trimming in this figure and the one shown in FIG. 2E is to secure the bottom of the ‘A’ lines so the pocket the ‘A’ lines create are closed enough to hold the cooling/warming material, also known as the non toxic gel pack.

FIG. 2G represents the back view of the two pieces of trimming that is sewn to the left and right top parts of FIG. 2A. They are both approximately 19 inches long and approximately 1 inch wide.

FIG. 2H represents side trimming that is sewn to the left sides of FIGS. 2B and 2C. It is approximately 19 inches long and approximately 1 inch wide.

FIG. 2I represents side trimming that is sewn to the right sides of FIGS. 2B and 2C. It is approximately 19 inches long and approximately 1 inch wide.

FIG. 2J represents the back view of the ‘A’ lines where the seat belts thread through. The material used to create the ‘A’ line is approximately 10 inches long and approximately 1 inch wide, but once the ‘A’ line is put into place, the opening itself is approximately 4 inches.

FIG. 2K represents the piece of material that is sewn onto the top middle part of FIG. 2A. It is approximately 9 and a half inches vertically and approximately 4 and a half inches horizontally. There is a piece of fastening tape on the top of it that connects to FIG. 1K. It is approximately 4 and a half inches horizontally and approximately 1 and a half inch vertically. Kozy Baby's main purpose is to cool or warm children when surrounding temperatures are not favorable. Kozy Baby can be put into many different seats used by children, such as a car seat or stroller, but it can also be used all by itself if one just needed an efficient way to cool or warm their child. This invention will solve many problems families have today regarding keeping their children comfortable.

1. A removable body pillow that is shaped to fit into a car seat or stroller that a child will lye against while properly seated in the car seat or stroller that has three rectangular pockets on the back of it where cooling/warming material, also known as non-toxic gel packs, are inserted to quickly and efficiently cool or warm the body of the child.

2. The removable body pillow, as described in claim 1, covers the majority of the area of the car seat or stroller where the child's body will lye against while properly seated in the car seat or stroller.

3. The removable body pillow, as described in claim 1, has three rectangular-shaped pockets located on the back of it, one located behind the area where the head of the child will lye against, another located behind the area where the back of the child will lye against, and a third located behind the area where the legs of the child will lye against. These pockets are where the cooling/warming material, also known as non-toxic gel packs, are inserted.

4. The removable body pillow, as described in claim 1, has an area on the top, frontal side of it where an additional neck support pillow, that is in the shape of an upside down letter “U”, can be attached if needed.

5. A pillow that provides comfort to a child while properly seated in a car seat or stroller when cooling/warming material, also known as non-toxic gel packs, are inserted, whether the non-toxic gel packs are cooled/warmed or not. It can be also described as therapeutic.

6. The removable body pillow, as described in claim 1, connects to the car seat or stroller easily with the use of a fastening tape, known on the market today as Velcro, so it is easily removed from the car seat or stroller.

7. The removable body pillow, as described in claim 1, includes an opening where the belt buckle of the car seat or stroller can easily fit through so the child is always secure in the seat.
